(19) United States
(12) Patent Application Publication
(10) Pub. No.: US 2024/0358543 A1
(43) Pub. Date: Oct. 31, 2024

(54) PELTIER-INTEGRATED THERAPEUTIC WRAP

(71) Applicant: UNIVERSITY OF NORTH TEXAS, Dallas, TX (US)

(72) Inventors: Kaleigh RUIZ, Dallas, TX (US); Samantha RYAN, Dallas, TX (US); Sarah STUTSMAN, Dallas, TX (US); Tanya TIRUMALA, Dallas, TX (US); JaTara WILLIAMS, Dallas, TX (US); Amir JAFARI, Dallas, TX (US); Trevor W. EXLEY, Dallas, TX (US)

(21) Appl. No.: 18/649,757

(22) Filed: Apr. 29, 2024

Related U.S. Application Data
(60) Provisional application No. 63/499,128, filed on Apr. 28, 2023.

(51) Int. Cl. A61F 7/00

(52) U.S. Cl. CPC A61F 7/007; A61F 2007/0035; A61F 2007/0075; A61F 2007/0082; A61F 2007/0095

(57) ABSTRACT

A device includes a flexible Peltier; a power supply in electrical communication with the flexible Peltier; a control switch electrically coupled to the power supply and the flexible Peltier. The control switch is configured to control direction of current supplied from the power supply to the flexible Peltier, and at least one flexible layer is coupled to the flexible Peltier. The at least one flexible layer is configured to retain the flexible Peltier about a portion of a subject.

### CROSS-REFERENCE TO RELATED APPLICATIONS

This application claims the benefit of U.S. Provisional Application No. 63/499,128, filed on Apr. 28, 2023, and entitled “OVERVIEW OF PELTIER-INTEGRATED THERAPEUTIC WRAP”, which is incorporated herein by reference in its entirety for all purposes.

### STATEMENT REGARDING FEDERALLY SPONSORED RESEARCH OR DEVELOPMENT

This invention was made with government support under contract no. 2213263 awarded by The National Science Foundation. The government has certain rights in the invention.

### BACKGROUND

Muscle and joint injury due to strain is a common occurrence among a variety of demographics. Muscle soreness, joint injury, and chronic joint conditions can be debilitating, affecting one's ability to perform daily activities. About 23.4 million people experience body pain or aches such as carpal tunnel, tendonitis, or arthritis, resulting in the use of various products such as ice packs, heating pads, roll-ons, or sprays. This condition is such a recognized issue that numerous devices have been designed to treat this pain, although current devices suffer from several shortcomings.

Examples of these shortcomings include that an ice pack requires to be frozen before use, melts over time, and can be discomforting for the user. A heating pad, such as a microwavable heating pad, can require a user to manually heat and can become too hot, and then may require re-heating. An electric heating pad only produces hot temperatures and usually requires a user to hold the pad against the affected area. Roll-ons, sprays, and other mendicants can result in skin irritation, require re-application, and have no temperature control.

Thus, there is a need for a device that overcomes these shortcomings of existing products to help a large population of individuals suffering, e.g., from joint pain and muscle soreness.

### SUMMARY

In some embodiments, a device includes a flexible Peltier; a power supply in electrical communication with the flexible Peltier; a control switch electrically coupled to the power supply and the flexible Peltier. The control switch is configured to control direction of current supplied from the power supply to the flexible Peltier, and at least one flexible layer is coupled to the flexible Peltier. The at least one flexible layer is configured to retain the flexible Peltier about a portion of a subject.

In some embodiments, a method of treating a patient comprises coupling a thermal treatment device to a subject, retaining the thermal treatment device about a portion of a subject using the at least one flexible layer, controlling direction of current supplied from the power supply to the flexible Peltier using the control switch, and applying heat or cooling to the subject based on the controlling. The thermal treatment device comprises a flexible Peltier, a power supply in electrical communication with the flexible Peltier, a control switch electrically coupled to the power supply and the flexible Peltier, and at least one flexible layer coupled to the flexible Peltier.

These and other features will be more clearly understood from the following detailed description taken in conjunction with the accompanying drawings and claims.

### BRIEF DESCRIPTION OF THE DRAWINGS

For a more complete understanding of the present disclosure, reference is now made to the following brief description, taken in connection with the accompanying drawings and detailed description:

FIG. 1 is a perspective, pictorial view of an embodiment of a device on a portion of a user.

FIG. 2 is a schematic depiction of an embodiment of a device.

FIG. 3 is an exploded, schematic view of an embodiment of a device.

FIG. 4 is a perspective, pictorial view of an embodiment of a flexible Peltier.

FIG. 5 is a perspective, pictorial view of an embodiment of a rigid Peltier.

FIG. 6 is a perspective, schematic view of an embodiment of an operational device.

FIG. 7 is a top, plan schematic view of an embodiment of a wrap.

FIG. 8 is a perspective, schematic view of an embodiment of at least one flexible layer of a wrap.

FIG. 9 is a top, schematic view of an embodiment of an electrical housing.

FIG. 10 is a perspective, schematic view of an embodiment of the electrical housing.

FIG. 11 is a right-side, schematic view of an embodiment of the electrical housing.

FIG. 12 is a left-side, schematic view of an embodiment of the electrical housing.

FIG. 13 is a side, schematic view of an embodiment of the electrical housing.

FIG. 14 is a perspective, pictorial view of an embodiment of the electrical housing.

FIG. 15 is a schematic view of an embodiment of a circuit connection for a wrap.

FIG. 16 is a schematic view of an embodiment of a linear semiconductor configuration.

FIG. 17 is a schematic view of an embodiment of a zig-zag semiconductor configuration.

### DETAILED DESCRIPTION

As used herein, the term “and/or” can mean one, some, or all elements depicted in a list. As an example, “A and/or B” can mean A, B, or a combination of A and B.

In some embodiments, a process is disclosed for utilizing the technology behind a specific flexible thermoelectric device called a Peltier that has the ability to both heat and cool. This concept can be applied to the joints affected by injuries or chronic conditions. The therapeutic wrap design can be applied to the joints most commonly affected by injuries and chronic conditions. For this device, mechanical properties as well as thermal management principles are implemented into a size-adjustable and joint-conforming therapeutic wrap that constantly monitors temperature conditions while in use. After wrapping the device around the affected joint or limb, users can easily switch between two modes—heating or cooling—by the flip of a switch. After wrapping the device around the affected, e.g., a joint, users can easily decide their preference between which two modes, such as heating or cooling, by flipping the switch. Thermal management and mechanical properties of flexible Peltiers were researched thoroughly before being implemented as size-adjustable for a wide range of users. Moreover, the joint-conforming therapeutic wrap can constantly monitor thermal conditions while in use.

Peltiers have many different applications in mechanical systems such as refrigerators, dehumidifiers, and satellites. These applications usually only utilize Peltiers for either heating or cooling, rarely using both in conjunction. Currently there are not any devices that utilize Peltiers in the medical field, specifically in therapeutics. Peltiers could be a unique replacement for these products, combining heating and cooling in a single device. Flexible Peltiers are a new technology, allowing multi-directional bending that helps the device conform to curved surfaces while still retaining its electrical properties. In some embodiments, flexible Peltiers can be used as a possible replacement for rigid Peltiers. These devices have the potential to be applied to a wide range of new applications, such as clothing or medical devices that require a high degree of flexibility and elasticity. Flexible Peltiers can be implemented into different applications such as in a mattress, a wearable wristband or device, waste heat utilization of industrial heat pipes, and other suitable applications. In some embodiments, a double flexible Peltier can be modified differently.

Temperature therapies are a common treatment for joint pain and muscle soreness. The application of cold temperatures in the range of about 5° C.-about 35° C., about 10° C.-about 30° C., about 15-about 25° C., or about 18° C.-about 24° C. are used for vasoconstriction, in other words restriction of blood flow, to lower inflammation and to induce analgesia following acute soft tissue injuries. The application of hot temperatures in the range of about 30° C.-about 55° C., about 35° C. to about 50° C., about 41° C.-about 45° C., or about 42° C.-about 43° C. are used to promote vasodilation, in other words to promote blood flow, soothe muscle tension, and treat muscle fiber injuries. The Peltier-integrated therapeutic wrap intended for joints such as the wrist or other limb(s), utilizes a unique thermoelectric device to maintain a heating temperature range of about 42° C.-about 43° C., and a cooling temperature range of about 18-about 24° C., both lying in the ranges of typical temperature therapies used currently. Thus, a user can switch between three modes, such as heating, cooling, and off.

Flexible Peltiers, otherwise known as thermoelectric coolers, are elastic electronics that generate high temperatures on one side, while producing low temperatures on the other side. The heating and cooling can be reversible. Rigid Peltiers—which are non-elastic—have many different applications in various systems such as in refrigerators, dehumidifiers, and satellites, and are commonly used for their cooling capability. However, there have not been any devices that utilize Peltiers in therapeutics. The integration of flexible Peltiers into a therapeutic wrap, is a new and exciting technology that allows users to choose between either the heating or cooling capabilities of the Peltier, depending on the pain they are experiencing.

Temperature hysteresis is produced by oscillating between low and high current. Temperature hysteresis can be applied to control the temperature range, produce lower temperatures, prevent overheating, and keep a user comfortable. Through the use of temperature hysteresis-applying an alternating current to the device, implemented with a timer relay, the flexible Peltier sustains the low-temperature range for a time period of about 1-about 5 minutes, sometimes about 3 minutes, with about 10-about 60-second period, sometimes about a 30-second period where the user must turn the device off and a maximum recommended cycle time of about 10-about 20 minutes, sometimes about 15 minutes. The heating mode of the therapeutic wrap is maintained using a temperature sensor programmed to turn off when the device reaches about 43° C., and turn back on when the temperature lowers to about 42° C. The hot temperatures can be maintained for a long period of time; however, it is only recommended for the user to use the heating function for about 20 minutes per some recommendations for the time period to apply heat to the skin. The materials used in this device can be selected by heat transfer, temperature, and tensile analysis to meet criteria such as heat dispersal, structure and fit, and user protection. Graphite and a silicone thermal pad can be used as a method of heat dispersal for when the Peltier is being used for its cooling function, as the device still produces hot temperatures on the other side. The thermal pad can decrease the external temperature of the wrap and assist in removing excess heat. An elastic bandage and nylon can be used to contain the Peltiers and ensure the device can stretch and conform to the user's joint while not restricting the user's circulation. The Peltiers can be placed in a double-layer configuration where both Peltiers are on at the same time, and the top Peltier can serve as an active heat sink for the Peltier that is closest to the skin. A heat sink can effectively store energy and can be used to sustain temperatures. This double-layer or stacked configuration produces a greater temperature difference between the heating and cooling sides of the Peltiers, and enables the device to maintain the heating and cooling temperature ranges for an extended period of time.

In some embodiments, a device can include an alternative current to direct current power adapter, such as twelve volt adapter, a double-pole, double-throw (DPDT) switch, a temperature sensor, two voltage regulators, and a timer relay. These components can be enclosed in a housing made from any suitable material, such as a polylactic acid, except for a flexible Peltier. Stacking two Peltiers can produce a greater temperature difference between the heating and/or cooling sides, and the top Peltier can act as an active heat sink. Temperature range of flexible Peltiers and how different materials interact with the flexible Peltiers can affect performance. In some embodiments, controls can be modified to provide a user with greater control of temperature selection.

In some embodiments, the Peltier-integrated therapeutic wrap can combine heating and cooling into one device. The device may combine temperature, material, and mechanical analysis to create effective thermal management of the one or more Peltiers. In some embodiments, more advanced user control over temperature selection and different configurations of semiconductors and copper pads can improve overall product elasticity.

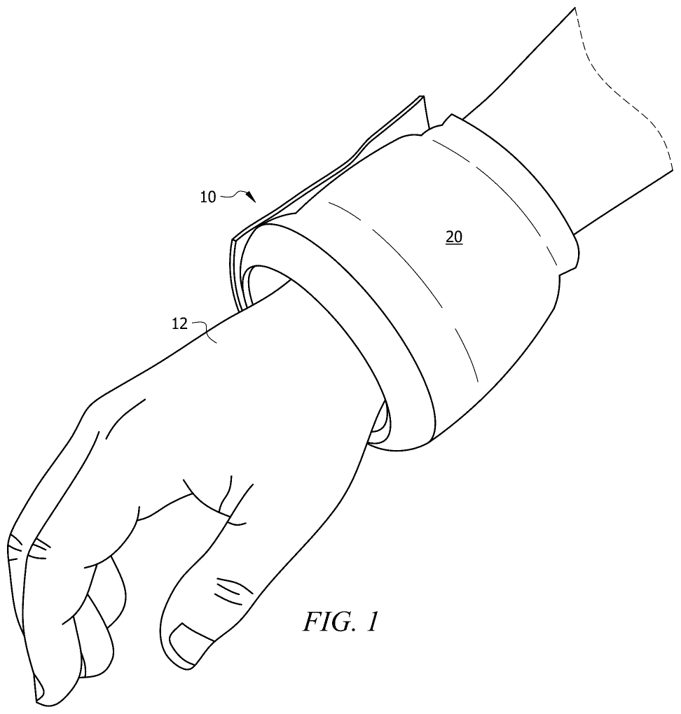

In some embodiments, as depicted in FIG. 1, a device 20 for treating muscle or joint injury can be placed on a subject 10, particularly a portion 12 of the subject 10. The portion 12 can be any part of the subject 10, including a limb and/or a joint such as a wrist or an ankle. The device 20 can be wrapped on the subject 10, as discussed below.

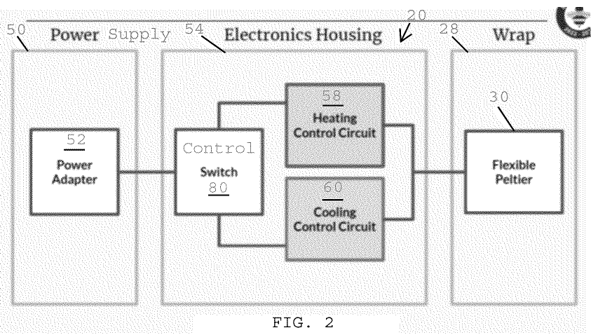

A schematic of some embodiments of the device 20 is depicted in FIG. 2. The device 20 can include a power supply 50, a housing 54, such as an electronics housing 54, and a wrap 28. The electronics housing 54 can include a control switch 80, a heating control circuit 58, and a cooling control circuit 60, and the wrap 28 can include a flexible Peltier 30. The power supply 50 can pass current through the power adapter 52 to, e.g., convert alternate current to direct current, to be communicated to the control switch 80 for passing through either through the heating control circuit 58 to emit heat or the cooling control circuit 60 to provide cooling via the flexible Peltier 30. The control switch 80 can be electrically coupled to the power supply 50 and the flexible Peltier 30. The control switch 80 is configured to control the direction of current supplied from the power supply 50 to the flexible Peltier 30.

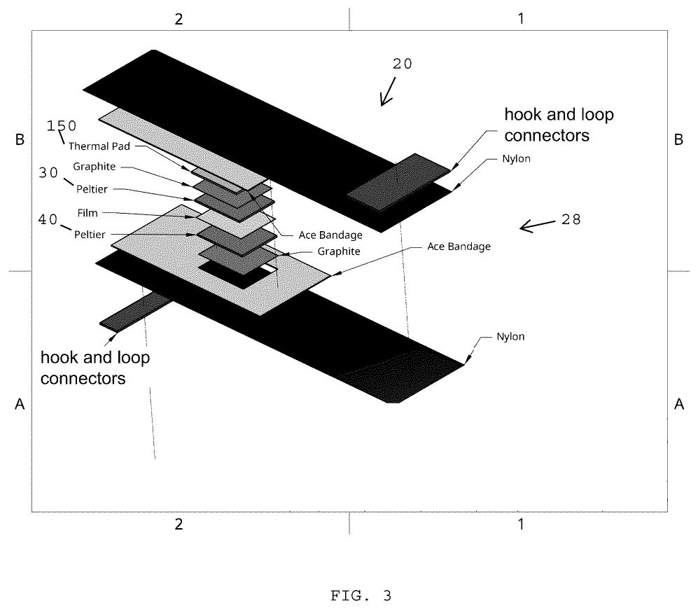

In some embodiments, as depicted in FIG. 3, a device 20 can include multiple components in the form of several layers. The wrap 28 can include loop and hook connectors, sold under trade designation VELCRO® by Velcro IP Holdings LLC, nylon, a thermal pad 150, an elastic bandage sold under the trade designation ACE™ by 3M Company, of St. Paul, Minnesota, graphite, a flexible Peltier 30 or a flexible Peltier 30, a film, and a second flexible Peltier 40. In some aspects, the first flexible Peltier 30 and the second flexible Peltier 40 can be stacked with or without the film therebetween. The thermal pad 150 can be coupled to the at least one flexible layer. The thermal pad 150 may be configured to conduct heat to and from the flexible Peltier 30. Referring FIGS. 4-5, respectively, a flexible Peltier 30 is depicted and a rigid Peltier 32 is depicted. In some embodiments, the flexible Peltier 30 and the rigid Peltier 32 may be interchangeable. In some embodiments, the flexible Peltier 30 is utilized. In some embodiments, referring to FIG. 6, a device 20 is depicted in operation having a cold side 22 having a temperature in a range of about 15-about 25° C. and a hot side 24 having a temperature in a range of about 41-about 45° C. Particularly, heat can be absorbed on the cold side 22 and heat rejected on the hot side 24. The device 20 may further include a thermocouple.

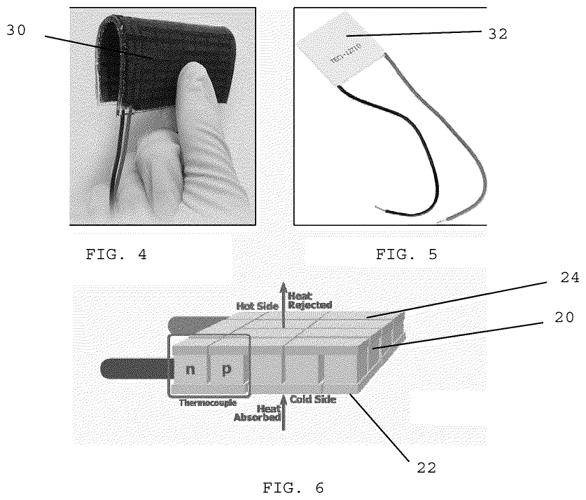

In some embodiments, referring to FIG. 7, the device 20 can include the wrap 28, and the first flexible Peltier 30 and the second flexible Peltier 40 incorporated into a double Peltier. This device 20 can be the wrap 28 for a portion 12, such as a wrist, of a subject 10, as depicted in FIG. 1.

In some embodiments, referring to FIG. 8, a wrap 28 is depicted with at least one flexible layer 90. Generally, the at least one flexible layer 90 is coupled to the flexible Peltier 30. The at least one flexible layer 90 is configured to retain the flexible Peltier 30 about the portion 12 of the subject 10, as depicted in FIG. 1. The wrap 28 can include a nylon exterior 62, an elastic bandage 64, an elastic bandage with a window 66, the thermal pad 150, first heat sink and second heat sink materials or layers 130, the first flexible Peltier 30, and the second flexible Peltier 40. The at least one heat sink material 130 or layer 130 may include graphite, configured to absorb or provide heat to the flexible Peltier 30. These layers 90 of the wrap 28 can be provided to a limb.

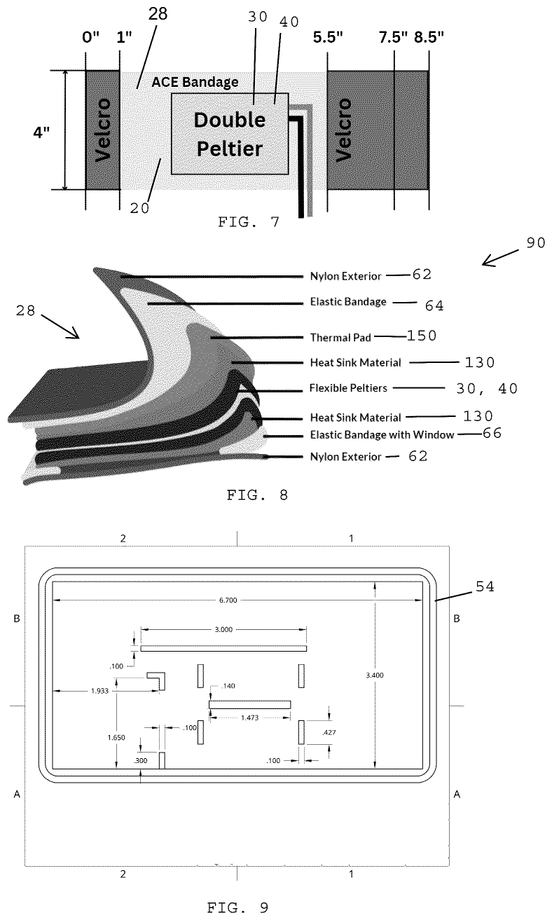

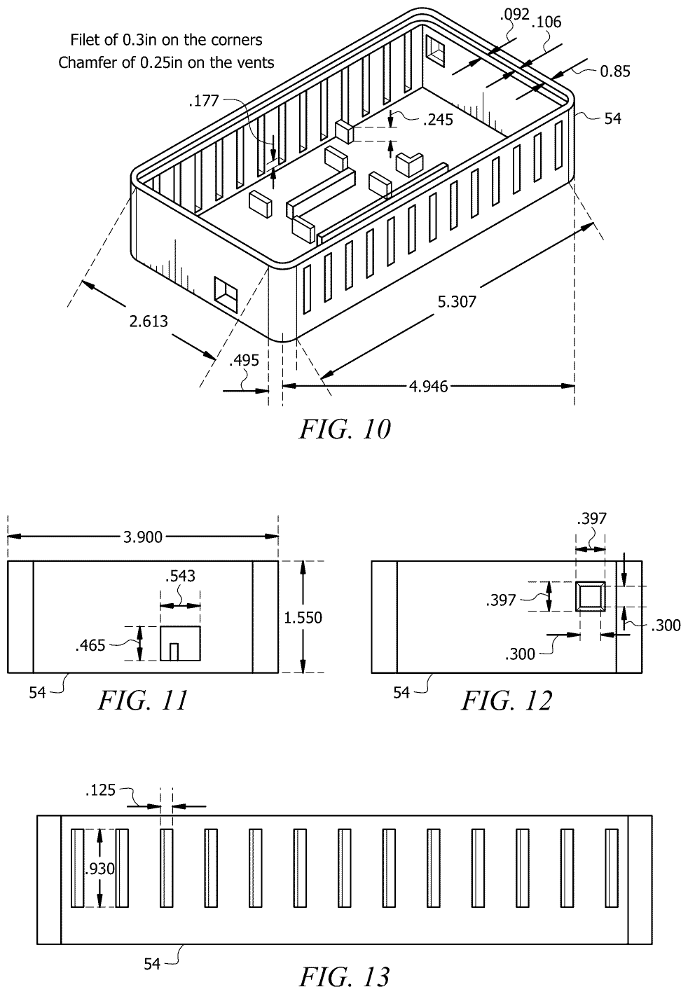

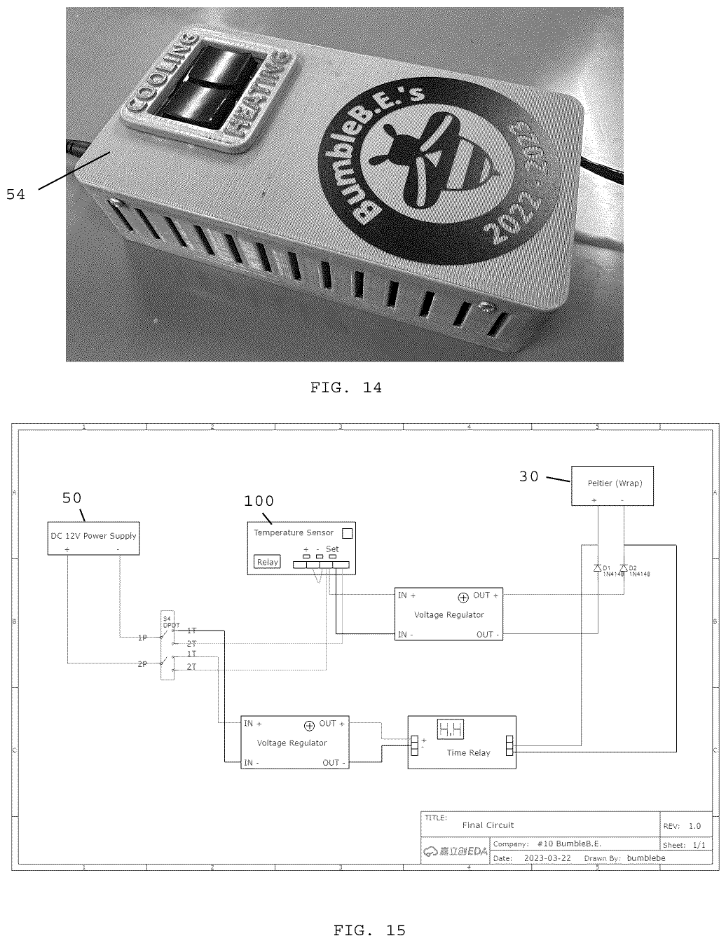

In some embodiments, referring to FIGS. 9-14, a housing 54, typically an electrical housing 54, can secure electrical components, such as the power adapter 52, the control switch 80, the heating control circuit 58, and the cooling control circuit 60, as depicted in FIG. 2.

In some embodiments, referring to FIG. 15, a circuit connection is used in the device 20 and is depicted having a direct current twelve volt power supply 50, a temperature sensor 100, and a Peltier 30 in a wrap 28. The circuit connection can further include two voltage regulators and a time relay. This circuit connection can be used to power the Peltier 30. The temperature sensor 100 is configured to control the application of current from the power supply 50 to the flexible Peltier 30.

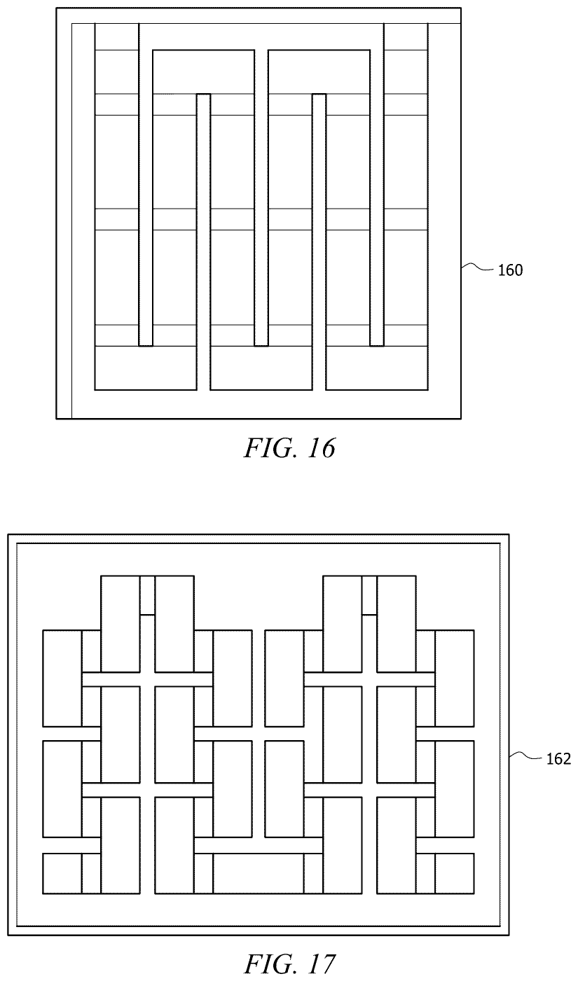

In some embodiments, referring to FIGS. 16-17, the semiconductors incorporated in the wrap 28 can have different configurations of semiconductors and copper pads to improve elasticity of the overall device 20. In some embodiments, referring to FIG. 16, the semiconductors can have a linear configuration 160, and referring to FIG. 17, the semiconductors can have a zig-zag configuration 162.

Having described various systems and methods herein, certain embodiments can include, but are not limited to:

In an aspect, a device comprises: a flexible Peltier; a power supply in electrical communication with the flexible Peltier; a control switch electrically coupled to the power supply and the flexible Peltier, wherein the control switch is configured to control direction of current supplied from the power supply to the flexible Peltier; and at least one flexible layer coupled to the flexible Peltier, wherein the at least one flexible layer is configured to retain the flexible Peltier about a portion of a subject.

A second aspect can include the device of the first aspect, further comprises a temperature sensor, wherein the temperature sensor is configured to control application of current from the power supply to the flexible Peltier.

A third aspect can include the device of the first aspect or the second aspect, further comprises: at least one heat sink layer, wherein the at least one heat sink layer comprises graphite.

A fourth aspect can include the device of any of the proceeding aspects, wherein the at least one heat sink layer is configured to absorb or provide heat to the flexible Peltier.

A fifth aspect can include the device of any of the proceeding aspects, further comprises a thermal pad coupled to the at least one flexible layer, wherein the thermal pad is configured to conduct heat to and from the flexible Peltier.

A sixth aspect can include the device of any of the proceeding aspects, further comprises a second flexible Peltier, wherein the flexible Peltier and the second flexible Peltier are stacked within the device.

A seventh aspect can include the device of any of the proceeding aspects, wherein the device is configured to generate a cold side having a temperature in a range of about 5° C.-about 35° C.

An eighth aspect can include the device of any of the proceeding aspects, wherein the device is configured to generate a cold side having a temperature in a range of about 15° C.-about 25° C.

A ninth aspect can include the device of any of the proceeding aspects, wherein the device is configured to generate a cold side having a temperature in a range of about 18° C.-about 24° C.

A tenth aspect can include the device of any of the proceeding aspects, wherein the device is configured to generate a hot side having a temperature in a range of about 30° C.-about 55° C.

An eleventh aspect can include the device of any of the proceeding aspects, wherein the device is configured to generate a hot side having a temperature in a range of about 41° C.-about 45° C.

A twelfth aspect can include the device of any of the proceeding aspects, wherein the device is configured to generate a hot side having a temperature in a range of about 42° C.-about 43° C.

For purposes of the disclosure herein, the term “comprising” includes “consisting” or “consisting essentially of.” Further, for purposes of the disclosure herein, the term “including” includes “comprising,” “consisting,” or “consisting essentially of.”

Accordingly, the scope of protection is not limited by the description set out above but is only limited by the claims which follow, that scope including all equivalents of the subject matter of the claims. Each and every claim is incorporated into the specification as an aspect of the present disclosure. Thus, the claims are a further description and are an addition to the aspects of the present invention. The discussion of a reference herein is not an admission that it is prior art to the presently disclosed subject matter, especially any reference that may have a publication date after the priority date of this application. The disclosures of all patents, patent applications, and publications cited herein are hereby incorporated by reference, to the extent that they provide exemplary, procedural or other details supplementary to those set forth herein.

While embodiments of the invention have been shown and described, modifications thereof can be made by one skilled in the art without departing from the spirit and teachings of the invention. Many variations and modifications of the subject matter disclosed herein are possible and are within the scope of the disclosed subject matter. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, etc.). Use of the term “optionally” with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, etc.

1. A device comprising:
a flexible Peltier;
a power supply in electrical communication with the flexible Peltier;
a control switch electrically coupled to the power supply and the flexible Peltier, wherein the control switch is configured to control direction of current supplied from the power supply to the flexible Peltier; and
at least one flexible layer coupled to the flexible Peltier, wherein the at least one flexible layer is configured to retain the flexible Peltier about a portion of a subject.

2. The device of claim 1, further comprising a temperature sensor, wherein the temperature sensor is configured to control application of current from the power supply to the flexible Peltier.

3. The device of claim 1, further comprising:
at least one heat sink layer, wherein the at least one heat sink layer comprises graphite.

4. The device of claim 3, wherein the at least one heat sink layer is configured to absorb or provide heat to the flexible Peltier.

5. The device of claim 1, further comprising a thermal pad coupled to the at least one flexible layer, wherein the thermal pad is configured to conduct heat to and from the flexible Peltier.

6. The device of claim 1, further comprising a second flexible Peltier, wherein the flexible Peltier and the second flexible Peltier are stacked within the device.

7. The device of claim 1, wherein the device is configured to generate a cold side having a temperature in a range of about 5° C.-about 35° C.

8. The device of claim 1, wherein the device is configured to generate a cold side having a temperature in a range of about 15° C.-about 25° C.

9. The device of claim 1, wherein the device is configured to generate a cold side having a temperature in a range of about 18° C.-about 24° C.

10. The device of claim 1, wherein the device is configured to generate a hot side having a temperature in a range of about 30° C.-about 55° C.

11. The device of claim 1, wherein the device is configured to generate a hot side having a temperature in a range of about 41° C.-about 45° C.

12. The device of claim 1, wherein the device is configured to generate a hot side having a temperature in a range of about 42° C.-about 43° C.

13. A method of treating a patient, the method comprising:
coupling a thermal treatment device to a subject, wherein the thermal treatment device comprises:
a flexible Peltier;
a power supply in electrical communication with the flexible Peltier;
a control switch electrically coupled to the power supply and the flexible Peltier,
at least one flexible layer coupled to the flexible Peltier,
retaining the thermal treatment device about a portion of a subject using the at least one flexible layer;
controlling direction of current supplied from the power supply to the flexible Peltier using the control switch; and
applying heat or cooling to the subject based on the controlling.

14. The method of claim 13, further comprising:
controlling application of current from the power supply to the flexible Peltier to control a temperature of the thermal treatment device.

15. The method of claim 13, wherein the thermal treatment device further comprises:
at least one heat sink layer comprises graphite, wherein the at least one heat sink layer is configured to absorb or provide heat to the flexible Peltier.

16. The method of claim 13, wherein the thermal treatment device further comprises: a thermal pad coupled to the at least one flexible layer, wherein the thermal pad is configured to conduct heat to and from the flexible Peltier.

17. The method of claim 13, wherein the thermal treatment device generates a cold side having a temperature in a range of about 5° C.-about 35° C.

18. The method of claim 13, wherein the wherein the thermal treatment device generates a cold side having a temperature in a range of about 15° C.-about 25° C.

19. The method of claim 13, wherein the wherein the thermal treatment device generates a hot side having a temperature in a range of about 30° C.-about 55° C.

20. The method of claim 13, wherein the wherein the thermal treatment device generates a hot side having a temperature in a range of about 41° C.-about 45° C.
